(19) United States
(12) Patent Application Publication
(10) Pub. No.: US 2004/0022826 A1
(43) Pub. Date: Feb. 5, 2004

(54) Bioartificial composite material and method for producing thereof

(76) Inventors: Gustav Steinhoff, Rethwisch (DE); Thomas Freier, Toronto (CA)

Correspondence Address: JORDAN AND HAMBURG LLP, 122 EAST 42ND STREET, SUITE 4000, NEW YORK, NY, 10168, US

(21) Appl. No.: 10/381,399

(22) PCT Filed: Sep. 20, 2001
(86) PCT No.: PCT/DE01/03625

(30) Foreign Application Priority Data
Sep. 25, 2000 (DE) 100473008

(51) Int. Cl. A61F 2/00

(52) U.S. Cl. 424/423; 424/426

(57) ABSTRACT

The aim of the invention is to provide transplants and a method for the production thereof, modelled on the natural product to such an extent that the above are well incorporated, do not lead to the possibility of rejection reactions, may thus be retained long-term, are considered as bodily material by the host and are replaced by the natural process of cell exchange such that the transplant may grow in the body of the recipient. The stability and the bio-compatibility of the transplant is increased by means of a coating method. According to the invention, said bioartificial composite material for tissue or organ replacement comprises an allogenic, xenogenic or autologous connective tissue matrix and/or a matrix of synthetic material, coated with polymers. Said material is applicable for heart valves, skin, vessels, aortas, tendons, corneas, cartilages, bones, tracheas, nerves, meniscuses, intervertebral discs, ureters, urethras and bladders.

### PRIOR ART

he invention relates to a method for the production of a bioartificial composite material using a connective tissue matrix which is coated with polymers.

Today, the transplantation of skin, vessels and organs is already surgically well controlled and widely used in the transplantation technique. However, it is still difficult to provide suitable transplants. The transplants can be classified according to their type into three major groups. For the moment, there are transplants and implants, respectively, made of artificial materials such as plastics, metals and ceramics, textile materials etc., according to the utilization and load resulting thereby. Secondly, it is possible to use allogenic material, e.g., donor organs or possibly xenogenous material (being of animal origin) as well. Finally, in some cases it is attempted to use endogenous tissue for a transplantation elsewhere.

method for the preparation of a tissue based on collagen for the transplantation is already known from U.S. Pat No. 5,336,616 which by means of the steps: Chemical pretreatment and cell removal, cryotreatment, dry stabilization, drying, rehydration and cell reconstitution is to enable an artificial transplant having the following characteristics: a) it includes an extracellular protein matrix which can be possibly remodelled and repaired by the host; b) it proves to be a membrane base in working order for the successful reseeding with viable endothelial or epithelial cells; c) it does not primarily induce any immunoresponse with the host; d) it does not calcify; and e) it can be simply stored and transported at ambient temperature.

The well-known method is based on the concept that a possibly “immunoneutral” supporting tissue matrix is to be formed as a transplant which can be reconstructed and remodelled then by the transplant recipient within the body. To facilitate this process the possibility of a subsequent seeding of the transplant produced according to the method is also provided. The disadvantage of this method is the high thrombogenicity of the collagen surface and a restriction of the mechanical stability.

A bioartificial recipient specific transplant and a method for producing a bioartificial transplant as well is described in WO 9900152 including the following steps: a) providing a native, allogenic or xenogenic tissue and material, respectively, containing a collagen matrix; b) largely removing all antigen-reactive cells from the collagen matrix by means of a careful cell removal, and subsequently rinsing with sterile aqueous solution; c) direct processing of the material made cell-free and loosened by the treatment according to b) by a possibly complete seeding with the respective desired autologous cells of the recipient or with genetically modified cells adapted for the recipient wherein a transplant being ready to immediately work is obtained. This transplant involves problems in so far as its structure is not stable enough in order to give a satisfactory solution with the transplantation in any case.

### OBJECT OF THE INVENTION

The object of the invention is to provide transplants and a method for the production thereof which are remodelled so far as on the natural material such that they grow into well, do not lead to rejection reactions (“Host-versus-graft reaction”) if possible, thus being durable for a long time, and are considered by the host as an endogenous material, and are reconstructed in the course of natural cell exchange such that even simultaneously growing of the transplants in the body of the recipient is possibly enabled. The stability and biocompatibility of the transplant are to be increased by means of a coating method.

The solution of this problem is achieved by means of a method for the production of a bioartificial composite material according to the invention for the tissue or organ replacement. With this, it is provided an allogenic, xenogenic or autologous tissue containing a connective tissue matrix and/or a matrix made of synthetic material. Processing of the material is accomplished by coating with polymers. The achieved matrix polymer composite material is stored in a sterile solution or in the solid state.

The synthetic matrix comprises proteins, peptides, carbohydrates or fats or the derivates thereof. The polymers are bioresorbable or non-resorbable.

Coating with polymers is accomplished by dipping into a polymer solution, subsequently removing the matrix from the solution and drying.

For processing from the solution with bioresorbable polymers, homopolymers such as poly(glycolide), poly(lactide), poly(ε-caprolactone), poly(trimethylene carbonate), poly(p-dioxanone), poly(hydroxybutyric acid) and longer chain poly(hydroxyalkanoates) are used as well as copolymers based on the mentioned polymers, which are used in a pure form or as blends. For the solvents, dichloromethane, trichloromethane, 1,2-dichloroethane, trichloroethylene, dibromomethane, dioxane, tetrahydrofuran, ethyl acetate, acetone, methyl ethyl ketone, acetonitrile, trifluoroethanol, hexafluoroaceton-sesquihydrate and hexafluoroisopropanol both in the pure form and as a mixture are used as low-boiling compounds.

For processing from the solution with non-resorbable polymers, poly(ethylene terephthalate), poly(urethane), silicone, poly(methacrylate), poly(sulfone), poly(ether sulfone), poly(ether ether ketone) or poly(carbonate) are used. Additionally, pentane, hexane and heptane, cyclohexane, benzene, toluene, diethylether and dimethylformamide are applied as solvents in addition to the ones mentioned with the resorbable polymers.

The bioresorbable and non-resorbable polymers can also be used as a blend to one another and/or with protein and/or with polysaccharides.

Coating with polymers can be accomplished by dipping into a polymer melt, subsequently removing the matrix from the melt and cooling.

For processing from the melt, poly(ε-caprolactone), poly(hydroxyalkanoates) and poly(p-dioxanone) having melting points below 150° C. or copolymers based on poly(ε-caprolactone), poly(hydroxyalkanoates) and poly(p-dioxanone) as well as leading from these with poly(glycolide) and poly(lactide) having melting points below 150° C. are applied as resorbable polymers.

The used solution or melt is also allowed to include additives besides the polymer which are plasticizers, contrast agent or active substances which are present in a soluble form or as a suspension.

The required layer thickness is adjusted by the number of dipping actions and the solution or melting viscosities. For coating spraying techniques both from the polymer solution and from the polymer melt can also be applied.

The bioartificial composite material for tissue or organ replacement comprises an allogenic, xenogenic or autologous connective tissue matrix and/or a matrix made of synthetic material which are coated with polymers.

As a soft-tissue replacement the most different applications in various organ systems are used:

a) skin and subskin tissues

b) blood vessels, heart tissues, heart valves

c) digestive system

d) urogenital system

e) respiratory system

f) parenchymal organs

g) muscles, ligaments and tendon tissues

h) neural replacement and peripheral nerves,

i) artificial extracorporeal and intracorporeal vessel systems

k) in-vitro reconstitution of organs and organ portions.

The composite material which is achieved in accordance with the method can be immediately applied. The storage and transportation should take place in a highly careful manner under sterile conditions.

### EXAMPLES

Matrix Materials

In the following, the invention will be described according to examples which include the isolation protocols for the isolation of autologous cells, and examples for carrying out the method.

An allogenic, xenogenic and autologous material, which is to provide the basic structure for the desired tissue, vessel or organ, serves as a starting material. In the individual case, here an autologous material can also be used which has been previously removed from the later transplant recipient. The starting material can be used for coating, cell containing as well as without any cells. According to the invention, the decellularization of this starting material is to be exclusively achieved by a careful cell removal and subsequently substantial rinsing with sterile aqueous solution if necessary. The careful removal of cells can be achieved either by careful enzymatic cell digestion such as by inserting the tissue and material, respectively, in a trypsin solution or alternatively by means of a chemically cell detaching agent, preferably by means of an ion specific complexing agent.

In the case of the enzymatic cell detachment, connective locations in the anchorage of the cells with the environment thereof are detached by the digestive enzyme. Preferably, this happens by inserting the tissue and material, respectively, in a trypsin solution. If required, EDTA, EGTA, triton or TNN can be added to this trypsin solution. By adjusting the duration during which the enzyme is acting on the tissue the amount of off-digestion is controlled, and it will be avoided that an attack to the collagen matrix takes place itself. The trypsin treatment leads to a good loosening of the matrix being made cell-free such that the new seeding is facilitated.

Alternatively, a chemically cell detaching agent is used which releases the cells on their anchorage to the collagen matrix in any other chemical manner. Preferably, it is provided that an ion specific complexing agent will be used which removes essential ions from the cells, and thus causing a detachment. By complexing of calcium ions, e.g., the interconnection of the cells one to another, and the cell matrix linkage through integrins are eliminated.

For example, the calcium specific EGTA [ethylene glycol-bis-(2-aminoethylether)-tetraacetic acid] can be used as an ion specific complexing agent. EGTA is preferably used in a short time and a high concentrated manner. Another complexing and chelating agents, respectively, such as EDTA (ethylenediaminetetraacetic acid), citrate or ethylenediamine or other chemically cell destroying and detaching agents which do not attack the collagen protein structure can also be used. Thus, there are further suitable: BAPTA/AM (a cell permeable calcium chelator), DMSO, gadolinium, desferrioxamine, desferrithiocine, hexadentate or aminocarboxylate as well. An advantage of the chemically mechanical treatment is in that the step of treatment in which the collagen matrix is made cell-free, needs to take up significantly less time only than during a treatment with trypsin. Thereby, firstly the danger decreases that the collagen matrix is attacked itself, and secondly the tertiary structure remains better maintained when the substrate will not be swallen too long in solution. Another advantage is in that by the application of “hard chemicals” problems can be avoided which are possibly caused by contaminated enzymes being of animal origin. Finally, a further advantage is in that the basal membrane remains in working order. The basal membrane represents the direct and tissue specific adhesion substrate for the endothelial cells.

The degree of glycolization of the matrix proteines also influences the cell immigration, and thus later immigrating of cells. The natural form of the collagen sceleton in its three-dimensional linkage with other matrix components and integrins can be largely maintained by means of the invention.

In the described manner, a loosened collagen structure is achieved, however, which still largely corresponds to the native polymerized collagen in its secondary and tertiary structures which is excellently prepared for the new cells to grow into, namely the autologous cells of the recipient or other immuno-tolerant cells if this is possible in the individual case.

As matrix materials, natural or synthetic proteins/peptides or polysaccharides can be used as well. Suitable member of the proteins/peptides are collagen, gelatin, elastin, fibronectin, laminin, polyaminoacids, e.g., as homopolymers or copolymers as well as oligomeric peptide units. From the polysaccharides, e.g., cellulose, hemicelluloses, pectin, marine polysaccharides such as alginate or carrageenan, microbial polysaccharides such as gellan, pullulan, glycosaminoglycans such as hyaluronic acid or dermatan sulfate, chitin/chitosan and storing polysaccharides such as starch or dextrane, and moreover derivates of the mentioned compounds are possible as matrix materials. The proteins/peptides and polysaccharides can be transformed into a matrix form according to the requirement by means of known techniques. The mechanical properties of the matrix can be adapted to the needs by means of chemical cross-linking reactions. Furthermore, it is possible to use blends of proteins/peptides and/or polysaccharides. Additives such as synthetic resorbable/non-resorbable polymers, microbial polyesters, plasticizers, contrast agent or active substances can be added to the proteins/peptides and/or polysaccharides. Proteins/peptides and polysaccharides can also be used for pretreatment of the tissue matrix wherein these are soaked with solutions or suspensions of proteins/peptides or/and polysaccharides. Here as well, the cross-linkage and addition of additives are possible.

Coating Materials

For coating the matrix materials, both resorbable and non-resorbable polymers are considered according to request. A precondition for coating the tissue, protein or polysaccharide matrix is a) for processing from the solution, the solubility of the polymer used for coating in a suitable solvent or b) for processing from the melt, a suitable melting temperature. Both coating from the polymer solution and coating from the polymer melt must not result in a change of the chemical structure of coating and matrix materials which influences the biological efficiency.

From the resorbable polymers, except from proteins/polypeptides and polysaccharides, homopolymers such as, e.g., poly(glycolide), poly(lactide), poly(ε-caprolactone), poly(trimethylene carbonate), poly(p-dioxanone), poly(hydroxybutyric acid) and longer chain poly hydroxyalkanoates) and copolymers based on the mentioned polymers as well are suitable for processing from the solution. The polymers and copolymers can be used in a pure form or as blends. Furthermore, the addition of additives such as plasticizers, contrast agent or active substances is possible.

As solvents for resorbable polymers, low-boiling compounds such as, e.g., dichloromethane, trichloromethane, 1,2-dichloroethane, trichloroethylene, dibromomethane, dioxane, tetrahydrofuran, ethyl acetate, acetone, methyl ethyl ketone, acetonitrile, trifluoroethanol, hexafluoroaceton-sesquihydrate and hexafluoroisopropanol both in a pure form and as a mixture can be used.

Generally, it is possible to influence the evaporation behaviour of the solvent during the coating process by a change of temperature.

For coating from the melt, resorbable polymers such as, e.g., poly(ε-caprolactone), poly(hydroxyalkanoates) and poly(p-dioxanone) having melting points below 150° C. are suitable. Copolymers based on poly(ε-caprolactone), poly(hydroxyalkanoates) and poly(p-dioxanone) as well as leading from these with poly(glycolide) and poly(lactide) having melting points below 150° C. are also considered for melt processing. The maximum temperature of the melt has to be adapted to the properties of the matrix in the individual case. With the use of the tissue or protein matrix a temperature of 80° C. is not to be exceeded.

Substantially, with the storage of the matrices coated with resorbable polymers the degradation kinetics has to be considered. This is allowed to result in a limited durability of the produced materials.

From the non-resorbable polymers except from proteins/polypeptides and polysaccharides, e.g., poly(ethylene terephthalate), poly(urethane), silicone, poly(methacrylate), poly(sulfone), poly(ether sulfone), poly(ether ether ketone) and poly(carbonate) can be used for processing from the solution. Additionally, compounds such as, e.g., pentane, hexane, heptane, cyclohexane, benzene, toluene, diethylether and dimethylformamide are used as solvents in addition to the compounds mentioned with the resorbable polymers.

Again, it is possible to influence the evaporation behaviour of the solvent during the coating process by a change of temperature. Also, the non-resorbable polymers can be used in a pure form or as blends with the addition of additives such as plasticizers, contrast agent or active substances.

Moreover, the proteins/peptides and polysaccharides are considered for coating. A stable coating requires the water insolubility thereof. For this, on the one hand there are provided proteins and polysaccharides being water-insoluble but soluble in other solvents or solvent mixtures also including water. On the other hand, water-soluble proteins and polysaccharides can be transformed into water-insoluble ones by chemical aftertreatment. The precondition is that the desired properties of the matrix will not be impaired. In addition to the mild chemical cross-linking reactions, both covalently and tonically, treatments with diluted acids or bases can be achieved.

The resorbable and non-resorbable polymers as well as the proteins and polysaccharides are allowed to be used as a mixture for the coating. For this, the same methods are provided as for the individual components.

Coating Methods

For coating from the solution there are required different methods depending on the used matrix material, solvents and polymers.

Generally, a solvent used for coating is to be added at least partially to the matrix stored in an aqueous solution prior to coating. This step can be eliminated with the use of water-soluble polymers. In the case of polymer solvents being mixable with water, the matrix has to be stored in this solvent for some minutes, and subsequently it can be immediately coated. In the case of solvents being not mixable with water, the matrix is to be stored in a solvent mixable with water for some minutes, and will be stored subsequently for some minutes in a solvent which is mixable with the preceding one. This procedure will be repeated until the polymer solvent can be added to the matrix. Subsequently, it is allowed again to coat immediately. During the exchange of solvent, in principle it is possible to add additives such as, e.g., dissolved active substances to the matrix.

In the case of a dry stored matrix adding with the polymer solvent is not required, it is allowed to coat immediately.

The actual polymer coating of the matrix is accomplished by short time dipping into the polymer solution, subsequently removing the matrix out of the solution, and drying. The required layer thickness can be adjusted by means of the number of dipping actions and viscosity of the solution. After drying the obtained matrix polymer composite material is stored in a sterile aqueous solution. The solution required for dipping may include additives such as plasticizers, contrast agent or active substances in addition to the polymer. These can be present in a dissolved form or as a suspension.

By means of dipping methods it is possible to generate porous layers in order to influence the cell growth. With this, an additive which can be removed after coating, e.g. by rinsing or thermal decomposition is added to the polymer solution. The additive can be present in the polymer solution in a dissolved or non-dissolved form. The porosity of the composite matrix to be generated can be adjusted through the additive/polymer ratio and particle size in case of unsoluble additives. By means of dipping methods it is further possible to generate layers which have a different composition. Thereby, active substances are allowed to selectively contact with the matrix material or the ambient environment or are allowed to be released at different times during the decomposition process of a resorbable polymer.

Coating from the polymer melt is accomplished with a dipping method which is analogous to the one being described for the polymer solution. Coating the matrix is accomplished by short time dipping into the polymer melt, subsequently removing the matrix from the melt, and cooling. The required layer thickness can be adjusted through the number of dipping actions and the melting viscosity. After cooling the achieved matrix polymer composite material is stored in a sterile aqueous solution. The polymer melt used for dipping can also include additives such as plasticizers, contrast agent or active substances. By means of dipping methods from the polymer melt it is possible again to generate porous layers. For this, an additive which can be removed after coating is added to the polymer melt. Furthermore, it is possible to generate layers which comprise a different composition. In addition to the described dipping method, spraying techniques both from the polymer solution and from the polymer melt can also be applied for coating.

### Embodiments

1. Matrix

A Matrix made of poly(β-hydroxybutyric acid)/gelatin:

Gelatin (0.1 g) is dissolved in formic acid (2.5 ml). Subsequently, trifluoroethanol (7.5 ml) is added. Thereafter, the addition of a solution of poly(β-hydroxybutyric acid) (0.4 g) in trifluoroethanol (10 ml) takes place by stirring. The solution of poly(β-hydroxybutyric acid)/gelatin thus achieved is poured into a matrix form. After a complete evaporation of the solvents a 1% (w/v) aqueous N′-3-dimethylaminopropyl-N-ethylcarbodiimide solution is added for 30 minutes. Subsequently, it is washed with water and stored in a sterile buffer solution.

A Matrix made of poly(β-hydroxybutyric acid)/chitosan:

Chitosan (0.1 g) is stirred in formic acid (2.5 ml) up to complete swelling. Subsequently, trifluoroethanol (7.5 ml) is added and stirred up to complete dissolution. Thereafter, the addition of a solution of poly(β-hydroxybutyric acid) (0.1 g) in trifluoroethanol (10 ml) takes place by stirring. The solution of poly(β-hydroxybutyric acid)/chitosan thus achieved is poured into a matrix form. After a complete evaporation of the solvents a 1% (w/v) aqueous caustic soda solution is added for 30 minutes. Subsequently, it is washed with water and stored in a sterile buffer solution.

A Matrix made of poly(β-hydroxybutyric acid)/gelatin/chitosan:

Gelatin and chitosan (0.1 g) each are stirred in formic acid (2.5 ml) up to complete swelling. Subsequently, trifluoroethanol (7.5 ml) is added and stirred up to complete dissolution. Thereafter, the addition of a solution of poly(β-hydroxybutyric acid) (0.1 g) in trifluoroethanol (10 ml) takes place by stirring. The solution of poly(β-hydroxybutyric acid), gelatin and chitosan thus achieved is poured into a matrix form. After complete evaporation of the solvents a 1% (w/v) aqueous caustic soda solution is added for 30 minutes. Subsequently, it is washed with water and a 1% (w/v) aqueous N′-3-dimethylaminopropyl-N-ethylcarbodiimide solution is added for 30 minutes. After washing with water it is stored in a sterile buffer solution.

2. Coating

Coating of Tissue Matrix with poly(β-hydroxybutyric acid):

A tissue matrix with a size of 1.0×0.5 cm is stored in acetone for 2×5 minutes. Subsequently, it is stored in chloroform for 5 minutes. After the chloroform storage the tissue matrix is dipped into a 1% (w/v) solution of poly(β-phydroxybutyric acid) in chloroform. After approximately 1 second, the matrix is removed from the polymer solution and dried in the air. Subsequently, it is stored in a sterile buffer solution.

Coating of Tissue Matrix with Gelatin:

A tissue matrix with a size of 1.0×0.5 cm is dipped into a 1% (w/v) aqueous gelatin solution. After approximately 1 second, the matrix is removed from the protein solution, and 1% (w/v) aqueous N′-3-dimethylaminopropyl-N-ethylcarbodiimide solution is added for 30 minutes. Subsequently, it is washed with water and stored in a sterile buffer solution.

Coating of Tissue Matrix with Poly(β-hydroxybutyric acid)/gelatin:

A tissue matrix with a size of 1.0×0.5 cm is stored in formic acid/trifluoroethanol (1:7) for two times 5 minutes. Thereafter, the tissue matrix is dipped into a 1% (w/v) solution of poly(β-hydroxybutyric acid)/gelatin (4:1) in formic acid/trifluoroethanol (1:7). After approximately 1 second, the matrix is removed from the polymer solution and dried in the air. Thereafter, a 1% (w/v) aqueous N′-3-dimethylaminopropyl-N-ethylcarbodiimide solution is added for 30 minutes. Subsequently, it is washed with water and stored in a sterile buffer solution.

Coating of Tissue Matrix with Chitosan:

A tissue matrix with a size of 1.0×0.5 cm is stored in 1% aqueous acetic acid for 2×5 minutes. Thereafter, the tissue matrix is dipped into a 1% (w/v) solution of chitosan in 1% aqueous acetic acid. After approximately 1 second, the matrix is removed from the polysaccharide solution, and a 1% (w/v) aqueous caustic soda solution is added for 30 minutes. Subsequently, it is washed with water and stored in a sterile buffer solution.

Coating of Tissue Matrix with poly(β-hydroxybutyric acid)/chitosan:

A tissue matrix with a size of 1.0×0.5 cm is stored in formic acid/trifluoroethanol (1:7) for 2×5 minutes. Thereafter, the tissue matrix is dipped into a 1% (w/v) solution of poly(β-hydroxybutyric acid)/gelatin (1:1) in formic acid/trifluoroethanol (1:7). After approximately 1 second, the matrix is removed from the polymer solution, and dried in the air. Thereafter, a 1% (w/v) aqueous caustic soda solution is added for 30 minutes. Subsequently, it is washed with water and stored in a sterile buffer solution.

Dipping from the Melt

A tissue matrix with a size of 1.0×0.5 cm is dipped into a melt of poly(γ-hydroxybutyric acid) which is heated to 60° C. After approximately 1 second, the matrix is removed from the polymer melt and stored in the air up to cooling and setting. Therafter, it is stored in a sterile buffer solution.

1. A method for the production of an artificial composite material, characterized in that
a) an allogenic, xenogenic or autologous tissue which includes a connective tissue matrix, and/or a matrix made of synthetic material are provided, and
b) processing the material is accomplished by coating with polymers
c) the obtained matrix polymer composite material is stored in a sterile solution or in the solid state.

2. A method according to claim 1, characterized in that said synthetic matrix comprises proteins, peptides, carbohydrates, fats or the derivates thereof.

3. A method according to claim 1 or claims 1 and 2, characterized in that said polymers are bioresorbable or not bioresorbable.

4. A method according to claim 3, characterized in that said coating with polymers is accomplished by dipping into a polymer solution, subsequently removing said matrix from said solution and drying.

5. A method according to claim 4, characterized in that for processing from the solution with bioresorbable polymers, as homopolymers are used poly(glycolide), poly(lactide) , poly(ε-caprolactone), poly(trimethylene carbonate), poly(p-dioxanone), poly(hydroxybutyric acid) and longer chain poly(hydroxyalkanoates) as well as copolymers based on the mentioned polymers which are used in a pure form or as blends.

6. A method according to claims 4 and 5, characterized in that dichloromethane, trichloromethane, 1,2-dichloroethane, trichloroethylene, dibromomethane, dioxane, tetrahydrofuran, ethyl acetate, acetone, methyl ethyl ketone, acetonitrile, trifluoroethanol, hexafluoroaceton-sesquihydrate and hexafluoroisopropanol both in a pure form and also as a mixture are used as low-boiling compounds for said solvents.

7. A method according to claim 4, characterized in that poly(ethylene terephthalate), poly(urethane), silicone, poly(methacrylate), poly(sulfone), poly(ether sulfone), poly(ether ether ketone) or poly(carbonate) are used for processing from said solution with said non-resorbable polymers wherein additionally pentane, hexane, heptane, cyclohexane, benzene, toluene, diethylether and dimethylformamide are applied as solvents in addition to said ones mentioned with said resorbable polymers.

8. A method according to claim 4, characterized in that said polymers used in the claims 5 and 7 are used as blend to one another, and/or with protein and/or with polysaccharides.

9. A method according to claim 3, characterized in that said coating with polymers is accomplished by dipping into a polymer melt, subsequently removing said matrix from said melt and cooling.

10. A method according to claim 9, characterized in that for processing from the melt poly(ε-caprolactone), poly(hydroxyalkanoates) and poly(p-dioxanone) having melting points below 150° C. or copolymers based on poly(ε-caprolactone), poly(hydroxyalkanoates) and poly(p-dioxanone) as well as leading from these with poly(glycolide) and poly(lactide) having melting points below 150° C. are applied as resorbable polymers.

11. A method according to any one of the claims 1 to 10, characterized in that
said used solution or melt also include additives in addition to said polymers which are plasticizers, contrast agent or active substances which are present in a dissolved form or as a suspension.

12. A method according to any one of the claims 1 to 11, characterized in that
the required layer thickness is adjusted by the number of dipping actions and the solution or melting viscosities.

13. A method according to any one of the claims 1 to 3, 5 to 8 or 10 to 12, characterized in that for coating spraying techniques both from said polymer solution and from said polymer melt can be applied.

14. A bioartificial composite material for tissue and organ replacement, characterized in that
said composite material comprises an allogenic, xenogenic or autologous tissue which includes a connective tissue matrix and/or a matrix made of synthetic material and is coated with polymers.

15. A use of an artificial composite material, characterized in that
most different applications in various organ systems are used as soft-tissue replacement:
a) skin and subskin tissues
b) blood vessels, heart tissues, heart valves
c) digestive system
d) urogenital system
e) respiratory system
f) parenchymal organs
g) muscles, ligaments and tendon tissues
h) neural replacement and peripheral nerves,
i) artificial extracorporeal and intracorporeal vessel systems
k) in-vitro reconstitution of organs and organ portions.
